(19) United States
(12) Patent Application Publication
(10) Pub. No.: US 2011/0027841 A1
(43) Pub. Date: Feb. 3, 2011

(54) METHOD FOR PREPARATION OF OXYLIPINS

(75) Inventor: Bindi Dangi, Elkridge, MD (US)

Correspondence Address: SHERIDAN ROSS PC, 1560 BROADWAY, SUITE 1200, DENVER, CO, 80202, US

(73) Assignee: MARTEK BIOSCIENCES CORPORATION, Columbia, MD (US)

(21) Appl. No.: 12/809,613

(22) PCT Filed: Dec. 22, 2008
(86) PCT No.: PCT/US08/87973

§ 371 Date: Oct. 14, 2010

Related U.S. Application Data
(60) Provisional application No. 61/016,235, filed on Dec. 21, 2007.

(51) Int. Cl. C12P 7/64

(52) U.S. Cl. 435/134

(57) ABSTRACT

The invention provides methods of producing oxygenated derivatives of polyunsaturated fatty acids (PUFAs).

### CROSS REFERENCE TO RELATED APPLICATION

This application claims the benefit of priority under 35 U.S.C. §119(e) to U.S. Provisional Patent Application Ser. No. 61/016,235 filed 21 Dec. 2007, which is incorporated herein by reference.

### TECHNICAL FIELD

The invention relates to optimized methods of producing oxygenated derivatives of polyunsaturated fatty acids (PUFAs) and more particularly, for the preparation of dihydroxy derivatives of long chain polyunsaturated fatty acids (LC-PUFAs).

### BACKGROUND OF INVENTION

Researchers in the 1990s identified hydroxy derivatives of some fatty acids in macroalgae (seaweeds) and described the possible role of these compounds in wound healing and cell signaling in the organisms (Gerwick & Bernart 1993; Gerwick et al 1993; Gerwick 1994). They recognized these compounds to be similar to those produced in the human body through the lipoxygenase pathway. These same researchers also attempted to develop cell suspension cultures of these seaweeds to produce eicosanoids and related oxylipins from C18 fatty acids (linoleic acid, and linolenic acid) and arachidonic acid (C20:4n-6) (ARA) in the red, brown and green seaweeds. However, production of seaweed biomass in these cultures systems proved to be very poor (e.g. about 0.6 to 1.0 g/L seaweed biomass after 15 days (Rorrer et al. 1996)) and even direct addition of key fatty acids to the cultures only minimally increased production of oxylipins over that of controls (Rorrer et al. 1997). Additionally, in some cases, the added free fatty acids proved toxic to the cultures (Rorrer et al. 1997). Therefore these systems have only remained academically interesting for producing oxygenated forms of these fatty acids, and studies continue on the C18 and C20 oxylipins in these seaweeds (e.g., Bouarab et al. 2004).

The oxylipins from the long chain omega-6 (n-6 or co-6 or N6) fatty acid, ARA, have been well studied and are generally considered to be proinflammatory in humans. Oxylipins from the long chain omega-3 (n-3 or ω-3 or N3) fatty acids, however, have generally been found to be anti-inflammatory. In the early 2000's, Serhan and other researchers discovered that hydroxylated forms of two long chain omega-3 polyunsaturated fatty acids (omega-3 LCPUFAs) (i.e., eicosapentaenoic acid (C20:5, n-3) (EPA) and docosahexaenoic acid C22:6, n-3) (DHA)) were made in the human body. They identified pathways whereby the omega-3 (n-3 or ω-3) LCPUFAs, EPA and DHA, were processed by cyclooxygenases, acetylated cyclooxygenase-2 or by lipoxygenase enzymes, resulting in production of novel mono-, di- and tri-hydroxy derivatives of these fatty acids. The resulting compounds, which were named “resolvins” (because they were involved in the resolution phase of acute inflammation) or docosatrienes (because they were made from docosahexaenoic acid and contain conjugated double bonds), were determined to have strong anti-inflammatory, antiproliferative, and neuroprotective properties. These compounds were also noted to have longer half-lives in the human body as compared to other types of eicosanoids.

In the past few years, various patents and patent application publications have described analogs of hydroxy derivatives of ARA, DHA and EPA, the pathways by which they are formed, methods for their synthesis in the laboratory via organic synthetic means or through biogenesis using cyclooxygenase or lipoxygenase enzymes, and use of these hydroxy derivatives as pharmaceutical compounds for the treatment of inflammatory diseases.

U.S. Patent Publication No. US-2006-0241088 A1 and U.S. Patent Publication No. US-2007-0248586-A1, both of which are incorporated herein by reference, describe novel oxylipins of various long chain omega-3 and omega-6 long chain polyunsaturated fatty acids, including docosapentaenoic acid (C22:5n-6; DPAn-6), docosapentaenoic acid (C22:5n-3; DPAn-3), docosatetraenoic acid (adrenic acid; C22:4n-6; DTAn-6), docosatetraenoic acid (C22:4n-3; DTAn-3) γ-linolenic acid (GLA; 18:3n-6), and stearidonic acid (STA or SDA; 18:4n-3).

However, there remains a need in the art for improved methods of producing oxylipins in commercially viable quantities and at lower cost.

### SUMMARY OF INVENTION

The invention provides methods of producing oxylipin derivatives of polyunsaturated fatty acids (LC-PUFAs). These methods include contacting a LC-PUFA substrate with sequential additions of an enzyme that catalyzes the production of the oxylipin derivatives from a PUFA substrate. In certain embodiments, the PUFA substrate is contacted with at least three or four sequential additions of the enzyme. In other embodiments, the PUFA substrate is contacted with at least five, or ten or fifteen or twenty sequential additions of the enzyme. In preferred embodiments, the PUFA substrate is contacted with about 2 to about 15 sequential additions of the enzyme. In preferred embodiments, the PUFA substrate is contacted with about 5 to about 12 sequential additions of the enzyme. In one preferred embodiment, the PUFA substrate is contacted with about 10 sequential additions of the enzyme. In another preferred embodiment, the PUFA substrate is contacted with a number of sequential additions of equal amounts of the enzyme sufficient to convert substantially all of the substrate to an oxylipin. In another preferred embodiment, the PUFA substrate is contacted with a number of sequential additions of equal amounts of the enzyme sufficient to convert at least 90% of the substrate to an oxylipin.

In an embodiment, the method includes adding an equal amount of enzyme in each sequential addition of enzyme. Similarly, an embodiment includes the sequential addition of enzyme at equal time intervals. In another embodiment, each sequential addition of the enzyme is contacted with the substrate about 20 minutes to about 45 minutes after the prior or first addition of enzyme. In another embodiment, the method includes the sequential addition of enzyme to contact the substrate for a time period sufficient to reduce the detectable enzyme activity to less than about 5%.

In some embodiments, the PUFA substrate may be one or more of di-homo-gammalinoleic acid (C20:3n-6), arachidonic acid (C20:4n-6), docosatetraenoic acid or adrenic acid (C22:4n-6), docosapentaenoic acid (C22:5n-6), docosadienoic acid (C22:2n-6), eicosatrienoic acid (C20:3n-3), eicosatetraenoic acid (C20:4n-3), eicosapentaenoic acid (C20:5n-3), docosatrienoic acid (C22:3n-3), docosapentaenoic acid (C22:5n-3), docosahexaenoic acid (C22:6n-3), C24:6(n-3), C28:8(n-3), γ-linolenic acid (18:3n-6) and stearidonic acid (18:4n-3). In one embodiment, the substrate is provided at a concentration of about 200 μM or less.

In some embodiments, the enzyme may be one or more of a lipoxygenase, a cyclooxygenase, and a cytochrome P450 enzyme. In related embodiments, the enzyme may be one or more of 12-lipoxygenase, 5-lipoxygenase, 15-lipoxygenase, cyclooxygenase-2, hemoglobin alpha 1, hemoglobin beta, hemoglobin gamma A, CYP4A11, CYP4B1, CYP4F11, CYP4F12, CYP4F2, CYP4F3, CYP4F8, CYP4V2, CYP4X1, CYP41, CYP2J2, CYP2C8, thromboxane A synthase 1, prostaglandin 12 synthase, and prostacyclin synthase.

In a specific embodiment, 10,17-diHDPAn-6 is produced by contacting DPAn-6 with 15-lipoxygenase, wherein the DPAn-6 is contacted with about 8 to 12 sequential additions of 15-lipoxygenase. In another specific embodiment, 10,17-diHDHA is produced by contacting DHA with 15-lipoxygenase, wherein the DHA is contacted with about 8 to 12 sequential additions of 15-lipoxygenase. In these specific embodiments, the enzyme additions may be made about every 25-35 minutes. In these specific embodiments, the amounts of enzyme added in each sequential addition of enzyme may be equal. The DPAn-6 or DHA may contacted with about 10 sequential additions of 15-lipoxygenase. The DPAn-6 or DHA may be provided at a concentration of about 200 μM or less.

### BRIEF DESCRIPTION OF DRAWING

FIG. 1, shows how the level of the monohydroxy derivative (absorbance at 237 nm) decreases while the level of the dihydroxy increases when the enzyme is added by sequential addition. The yield of oxylipin produced using this protocol was at least 4-5 fold higher than with conventional methods using significantly more substrate and a single addition of enzyme.

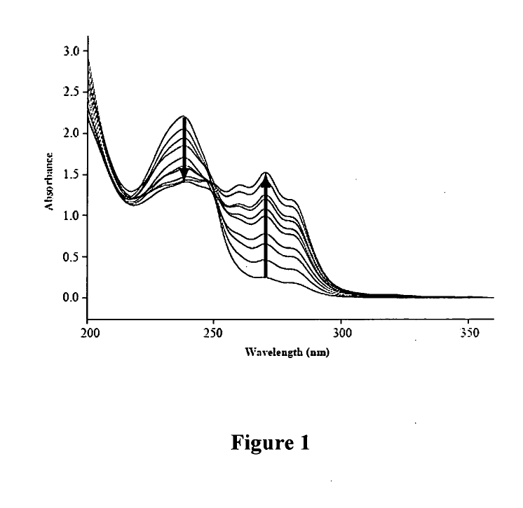

### DESCRIPTION OF EMBODIMENTS

The present invention generally relates to an optimized process for the preparation of oxylipins, and particularly, for the preparation of dihydroxy oxylipin derivatives of polyunsaturated fatty acids (PUFAs) and more particularly, for the preparation of dihydroxy oxylipin derivatives of long chain polyunsaturated fatty acids (LC-PUFAs). The method is exemplified by the preparation of 10,17-diHDPAn-6 (10S,17S-dihydroxy-docosa-4Z,7Z,10Z,13Z,19Z-pentaenoic acid), although the same protocol can be used for preparing dihydroxy derivatives from other LC-PUFAs. For example, 10,17-diHDHA (10S,17S-dihydroxy-docosa-4Z,7Z,11E,13Z,15E,19Z-hexaenoic acid; a Neuroprotectin D1 (NPD1) isomer) is obtained when DHA is used as a substrate. The process of the present invention is superior to protocols for producing oxylipins described prior to the invention. For example, prior processes use much higher concentrations of substrate (>500 μM in some cases) and the yields of oxylipins are typically only about 20-25% of the yields achieved using the method of the present invention.

More specifically, the present invention provides a method for converting a LC-PUFA substrate to an oxylipin derivative thereof by contacting the LC-PUFA with an enzyme that is capable of catalyzing the conversion. The method differs from methods described prior to the invention in that the inventor has discovered that if the enzyme is added to the substrate in three or more sequential additions, the oxylipin yields obtained are substantially higher than if the enzyme is added all at once (a single addition). Indeed, if the enzyme is added to the reaction all at once (wherein the amount of enzyme in the single addition is equal to the total amount of enzyme added when done sequentially in three or more additions), the same yields are not obtained and are at best at about 20-25% of the yield of enzyme obtained when using the method of the present invention. In addition, the present inventor has discovered that if substrate is provided in the method of the present invention at concentrations above about 200 μM, the oxylipin derivative is not formed in significant amounts. This is in contrast to methods described prior to the invention, in which much higher concentrations of substrate are used (e.g., >500 μM in some cases), which makes the method of the present invention substantially more efficient and cost-effective than previously described methods.

Accordingly, one embodiment of the invention relates to a method to produce oxylipin derivatives of long chain polyunsaturated fatty acids (LC-PUFAs). The method includes the step of catalytically producing the oxylipin derivatives by contacting an LC-PUFA substrate with an enzyme that catalyzes the production of the oxylipin derivative from the substrate. The method includes the contact of the substrate with at least three sequential additions of the enzyme during the course of the reaction. Preferably, the number of sequential additions of enzyme is sufficient to convert most of the substrate, and in one aspect, substantially all of the substrate, to its oxylipin derivative (e.g., at least 90%, and more preferably, about 100% of the substrate is converted to oxylipin during the course of the reaction). In one aspect, the enzyme is added to the substrate reaction in at least 3, 4, 5, 6, 7, 8, 9, 10, 11, 12, 13, 14, or 15 sequential additions. In another aspect, the enzyme is added to the substrate reaction in from about 3 to about 15 sequential additions of the enzyme. In another aspect, the substrate is contacted with from about 5 to about 12 sequential additions of the enzyme. In one preferred aspect, the substrate is contacted with about 10 sequential additions of the enzyme.

Preferably, the amount of enzyme added in each of the sequential additions of enzyme is equal to the others, although the amount of enzyme added in each sequential addition can be modified (increased or decreased) relative to the prior addition. Preferably, the total amount of enzyme added through all of the sequential additions of enzyme is approximately equal to or less than the amount of enzyme that would be added if the enzyme was added in a single addition to the reaction.

The timing between sequential additions of enzymes according to the invention is equal to the others, in one aspect of the invention, although as the reaction progresses and the substrate is converted, the timing may be modified to be more or less than that of the prior interval. Also, if the amount of enzyme added at each addition is modified, then the timing before adding the next addition can be modified to maximize the activity of the enzyme additions. The timing between enzyme additions is most preferably the time required to reduce most of the enzyme activity in one addition to undetectable levels, so that the maximum activity from each enzyme addition is utilized. Preferably, once this point is reached, the next enzyme addition is added, to avoid wasting time between enzyme additions. One of skill in the art will be able to determine the optimum timing between intervals based on the substrate and enzyme combination used, the amount of substrate in the reaction, and the amount of enzyme added at each interval. In one aspect, each sequential addition of the enzyme is contacted with the substrate for a time period sufficient to reduce the detectable enzyme activity to about 5% or less. In one aspect, each sequential addition of the enzyme is contacted with the substrate about 20 minutes to about 45 minutes after the prior or first addition of enzyme. In another aspect, each sequential addition of the enzyme is contacted with the substrate for about 30 minutes after the prior or first addition of enzyme.

The method of the invention can be used to convert any LC-PUFA or PUFA substrate to its oxylipin derivative. In one aspect, the LC-PUFA is selected from: di-homo-gammalinoleic acid (C20:3n-6), arachidonic acid (C20:4n-6), docosatetraenoic acid or adrenic acid (C22:4n-6), docosapentaenoic acid (C22:5n-6), docosadienoic acid (C22:2n-6), eicosatrienoic acid (C20:3n-3), eicosatetraenoic acid (C20:4n-3), eicosapentaenoic acid (C20:5n-3), docosatrienoic acid (C22:3n-3), docosapentaenoic acid (C22:5n-3), docosahexaenoic acid (C22:6n-3), C24:6(n-3), C28:8(n-3), γ-linolenic acid (18:3n-6) and stearidonic acid (18:4n-3). Oxylipin derivatives of these PUFAs are described in some detail below, and also in U.S. Patent Publication No. US-2006-0241088 A1 and U.S. Patent Publication No. US-2007-0248586-A1, both of which are incorporated herein by reference.

The enzyme to be used in the method of the invention can include any oxylipin-generating enzyme including, but not limited to lipoxygenases, cyclooxygenases, cytochrome P450 enzymes and other heme-containing enzymes, such as those described in Table 1 (see below). In one aspect, the enzyme is selected from: 12-lipoxygenase, 5-lipoxygenase, 15-lipoxygenase, cyclooxygenase-2, hemoglobin alpha 1, hemoglobin beta, hemoglobin gamma A, CYP4A11, CYP4B1, CYP4F11, CYP4F12, CYP4F2, CYP4F3, CYP4F8, CYP4V2, CYP4X1, CYP41, CYP2J2, CYP2C8, thromboxane A synthase 1, prostaglandin 12 synthase, and prostacyclin synthase.

Table 1 provides sufficient information to identify the listed known enzymes, including official names, official symbols, aliases, organisms, and/or sequence database accession numbers for the enzymes.

TABLE 1
Lipoxygenase (LOX), cyclooxygenase (COX), cytochrome P450 (CYP)
enzymes and other heme-containing enzymes that can be used to process LCPUFA oils
and fatty acids to produce their hydroxyl fatty acid derivatives by methods described
herein.

LIPOXYGENASE TYPE ENZYMES
ALOX12
Official Symbol: ALOX12 and Name: arachidonate 12-lipoxygenase [Homo
sapiens]
Other Aliases: HGNC: 429, LOG12
Other Designations: 12(S)-lipoxygenase; platelet-type 12-lipoxygenase/arachidonate 12-lipoxygenase
Chromosome: 17; Location: 17p13.1GeneID: 239
Alox5
Official Symbol: Alox5 and Name: arachidonate 5-lipoxygenase [Rattus
norvegicus]
Other Aliases: RGD: 2096, LOX5A
Other Designations: 5-Lipoxygenase; 5-lipoxygenase
Chromosome: 4; Location: 4q42GeneID: 25290
ALOXE3
Official Symbol: ALOXE3 and Name: arachidonate lipoxygenase 3 [Homo
sapiens]
Other Aliases: HGNC: 13743
Other Designations: epidermal lipoxygenase; lipoxygenase-3
Chromosome: 17; Location: 17p13.1GeneID: 59344
LOC425997
similar to arachidonate lipoxygenase 3; epidermal lipoxygenase; lipoxygenase-3 [Gallus gallus]
Chromosome: UnGeneID: 425997
LOC489486
similar to Arachidonate 12-lipoxygenase, 12R type (Epidermis-type lipoxygenase 12) (12R-lipoxygenase)
(12R-LOX) [Canis familiaris]
Chromosome: 5GeneID: 489486
LOC584973
similar to Arachidonate 12-lipoxygenase, 12R type (Epidermis-type lipoxygenase 12) (12R-lipoxygenase)
(12R-LOX) [Strongylocentrotus purpuratus]
Chromosome: UnGeneID: 584973
LOC583202
similar to Arachidonate 12-lipoxygenase, 12R type (Epidermis-type lipoxygenase 12) (12R-lipoxygenase)
(12R-LOX) [Strongylocentrotus purpuratus]
Chromosome: UnGeneID: 583202
LOC579368
similar to Arachidonate 12-lipoxygenase, 12R type (Epidermis-type lipoxygenase 12) (12R-lipoxygenase)
(12R-LOX) [Strongylocentrotus purpuratus]
Chromosome: UnGeneID: 579368
LOC504803
similar to Arachidonate 12-lipoxygenase, 12R type (Epidermis-type lipoxygenase 12) (12R-lipoxygenase)
(12R-LOX) [Bos taurus]
Chromosome: UnGeneID: 504803
ALOX5
Official Symbol: ALOX5 and Name: arachidonate 5-lipoxygenase [Homo sapiens]
Other Aliases: HGNC: 435, 5-LO, 5LPG, LOG5Other Designations: arachidonic acid 5-lipoxygenase;
leukotriene A4
synthaseChromosome: 10; Location: 10q11.2GeneID: 240
OSJNBa0057G07.
15 lipoxygenase L-2; lipoxygenase [Oryza sativa (japonica cultivar-group)]GeneID: 3044798
Alox15b
Official Symbol: Alox15b and Name: arachidonate 15-lipoxygenase, second type [Mus musculus]
Other Aliases: MGI: 1098228, 8-LOX, 8S-LOX, Alox8
Other Designations: 8S-lipoxygenase
Chromosome: 11; Location: 11 B4GeneID: 11688
ALOX5AP
Official Symbol: ALOX5AP and Name: arachidonate 5-lipoxygenase-activating protein [Homo sapiens]
Other Aliases: HGNC: 436, FLAP
Other Designations: MK-886-binding protein; five-lipoxygenase activating protein
Chromosome: 13; Location: 13q12GeneID: 241
LOC489485
similar to Arachidonate 15-lipoxygenase, type II (15-LOX-2) (8S-lipoxygenase) (8S-LOX) [Canis
familiaris]
Chromosome: 5GeneID: 489485
LOC557523
similar to Arachidonate 5-lipoxygenase (5-lipoxygenase) (5-LO) [Danio
rerio]
Chromosome: 15GeneID: 557523
Alox5ap
Official Symbol: Alox5ap and Name: arachidonate 5-lipoxygenase activating protein [Mus musculus]
Other Aliases: MGI: 107505, Flap
Other Designations: arachidonate 5 lipoxygenase activating protein
Chromosome: 5GeneID: 11690
LOC562561
similar to Arachidonate 5-lipoxygenase (5-lipoxygenase) (5-LO) [Danio
rerio]
Chromosome: UnGeneID: 562561
LOC423769
similar to Arachidonate 5-lipoxygenase (5-lipoxygenase) (5-LO) [Gallus
gallus]
Chromosome: 6GeneID: 423769
LOC573013
similar to Arachidonate 5-lipoxygenase (5-lipoxygenase) (5-LO) [Danio
rerio]
Chromosome: UnGeneID: 573013
LOC584481
similar to Arachidonate 5-lipoxygenase (5-lipoxygenase) (5-LO)
[Strongylocentrotus purpuratus]
Chromosome: UnGeneID: 584481
5LOX-potato
AAD04258. Reports 5-lipoxygenase [S . . . [gi: 2789652]
15-LOX Soybean
P08170. Reports Seed lipoxygenase . . . [gi: 126398]
12-LOX-porcine
D10621. Reports Sus scrofa gene f . . . [gi: 60391233]
B) CYCLOOXYGENASE ENZYMES
COX2-human
AAN87129. Reports prostaglandin syn . . . [gi: 27151898]
C) HEMOGLOBIN CONTAINING ENZYMES
HBA1
Official Symbol: HBA1 and Name: hemoglobin, alpha 1 [Homo sapiens]
Other Aliases: HGNC: 4823, CD31
Other Designations: alpha 1 globin; alpha one globin; alpha-1 globin; alpha-1-globin; alpha-2 globin; alpha-
2-globin; hemoglobin alpha 1 globin chain; hemoglobin alpha 2; hemoglobin alpha-1 chain; hemoglobin
alpha-2
Chromosome: 16; Location: 16p13.3GeneID: 3039
HBB
Official Symbol: HBB and Name: hemoglobin, beta [Homo sapiens]
Other Aliases: HGNC: 4827, CD113t-C, HBD, hemoglobin
Other Designations: beta globin; beta globin chain; haemoglobin A beta chain; hemoglobin beta chain;
hemoglobin delta Etolia variant
Chromosome: 11; Location: 11p15.5GeneID: 3043
HBG1
Official Symbol: HBG1 and Name: hemoglobin, gamma A [Homo sapiens]
Other Aliases: HGNC: 4831, HBGA, HBGR, HSGGL1, PRO2979
Other Designations: A-gamma globin; gamma A hemoglobin; gamma globin; hemoglobin gamma-a chain;
hemoglobin, gamma, regulator of
Chromosome: 11; Location: 11p15.5GeneID: 3047
D) CYTOCHROME P450 TYPE ENZYMES
(Gene, Organism, Gene Database: SwissProt, Gene database: EMBL/Genbank/DDBJ)
CYP4A11, Homo sapiens, CP4AB_HUMAN, L04751 D26481 S67580 S67581 AF525488 AY369778
X71480
CYP4A4, Oryctolagus cuniculus, CP4A4_RABIT, L04758 J02818
CYP4A5, Oryctolagus cuniculus, CP4A5_RABIT, M28655 X57209
CYP4A6, Oryctolagus cuniculus, CP4A6_RABIT, M28656 M29531
CYP4A7, Oryctolagus cuniculus, CP4A7_RABIT, M28657 M29530
CYP4B1, Homo sapiens, CP4B1_HUMAN, J02871 X16699 AF491285 AY064485 AY064486
CYP4B1, Oryctolagus cuniculus, CP4B1_RABIT, M29852 AF176914 AF332576
CYP4C1, Blaberus discoidalis, CP4C1_BLADI, M63798
CYP4C21, Blattella germanica, CP4CU_BLAGE, AF275641
CYP4E4, Drosophila melanogaster, C4AE1_DROME, AE003423 AL009194 AY058450 U34331
CYP4F11, Homo sapiens, CP4FB_HUMAN, AF236085 BC016853 AC005336
CYP4F12, Homo sapiens, CP4FC_HUMAN, AY008841 AB035130 AB035131 AY358977
CYP4F2, Homo sapiens, CP4F2_HUMAN, D26480 U02388 AB015306 AF467894 AC005336 BC067437
BC067439 BC067440 AF221943
CYP4F3 Homo sapiens CP4F3_HUMAN, D12620 D12621 AB002454 AB002461 AF054821 AY792513
CYP4F8 Homo sapiens CP4F8_HUMAN, AF133298
CYP4V2 Homo sapiens CP4V2 HUMAN, AY422002 AK122600 AK126473 BC060857
CYP4V2, Pongo pygmaeus CP4V2_PONPY, CR858234
CYP4X1, Homo sapiens CP4X1_HUMAN, AY358537 AK098065 BC028102
CYP4Z1, Homo sapiens CP4Z1_HUMAN, AY262056 AY358631
Cyp4a1, Rattus norvegicus CP4A1_RAT, M14972 X07259 M57718
Cyp4a2, Rattus norvegicus CP4A2_RAT, M57719 BC078684
Cyp4a3, Rattus norvegicus CP4A3_RAT, M33936
Cyp4a8, Rattus norvegicus CP4A8_RAT, M37828
Cyp4aa1, Drosophila melanogaster, C4AA1_DROME AE003808
Cyp4ac1, Drosophila melanogaster, C4AC1_DROME AE003609 AY051602
Cyp4ac2, Drosophila melanogaster, C4AC2_DROME, AE003609
Cyp4ac3, Drosophila melanogaster, C4AC3_DROME, AE003609 AY061002
Cyp4ad1, Drosophila melanogaster, C4AD1_DROME, AE003837 AY061058
Cyp4b1, Mus musculus, CP4B1_MOUSE, D50834 BC008996
Cyp4b1 Rattus norvegicus CP4B1_RAT, M29853 BC074012
Cyp4c3, Drosophila melanogaster, CP4C3_DROME, AE003775 BT010108 U34323
Cyp4d1, Drosophila melanogaster, CP4D1_DROME, X67645 AF016992 AF016993 AF016994 AF016995
AF016996 AF016997 AF016998 AF016999 AF017000 AF017001 AF017002 AF017003 AF017004
AE003423 AE003423 Z98269
Cyp4d1, Drosophila simulans, CP4D1_DROSI, AF017005
Cyp4d10, Drosophila mettleri, C4D10_DROMT, U91634
Cyp4d14, Drosophila melanogaster, C4D14_DROME, AE003423 AL009194
Cyp4d2, Drosophila melanogaster, CP4D2_DROME, X75955 Z23005 AE003423 AL009194 AY118763
AF017006 AF017007 AF017008 AF017009 AF017010 AF017011 AF017012 AF017013 AF017014
AF017015 AF017016 AF017017 AF017018-Cyp4d20, Drosophila melanogaster, C4D20_DROME,
AE003475
Cyp4d21, Drosophila melanogaster, C4D21_DROME, AE003618
Cyp4d8, Drosophila melanogaster, CP4D8_DROME, AE003558 AY058442 U34329
Cyp4e1, Drosophila melanogaster, CP4E1_DROME, AE003837 AY118793
Cyp4e2, Drosophila melanogaster, CP4E2_DROME, U56957 AE003837 AY058518 X86076 U34332
Cyp4e3, Drosophila melanogaster, CP4E3_DROME, AE003626 U34330
Cyp4e5, Drosophila mettleri, CP4E5_DROMT, U78486
Cyp4f1, Rattus norvegicus, CP4F1_RAT, M94548 AF200361
Cyp4f14, Mus musculus, CP4FE_MOUSE, AB037541 AB037540 AF233644 AK005007 AK018676
BC011228
Cyp4f4, Rattus norvegicus, CP4F4_RAT, U39206
Cyp4f5, Rattus norvegicus, CP4F5_RAT, U39207
Cyp4f6, Rattus norvegicus, CP4F6_RAT, U39208
Cyp4g1, Drosophila melanogaster, CP4G1_DROME, AE003417 AL009188 U34328
Cyp4g15, Drosophila melanogaster, C4G15_DROME, AF159624 AE003486 AY060719
Cyp4p1, Drosophila melanogaster, CP4P1_DROME, AE003834 AY071584 U34327
Cyp4p2, Drosophila melanogaster, CP4P2_DROME, AE003834 AY051564
Cyp4p3, Drosophila melanogaster, CP4P3_DROME, AE003834 AY075201
Cyp4s3, Drosophila melanogaster, CP4S3_DROME AE003498
Cyp4v3, Mus musculus, CP4V3_MOUSE, AB056457 AK004724
Cyp4x1, Rattus norvegicus, CP4X1_RAT, AF439343
CYP2 Family of Cytochrome P450 Enzymes (sequences from Genbank)
CYP2J2 sequences from GenBank
NM_000775
Homo sapiens cytochrome P450, family 2, subfamily J, polypeptide 2 (CYP2J2)
gi|18491007|ref|NM_000775.2|[18491007]
NM_000770
Homo sapiens cytochrome P450, family 2, subfamily C, polypeptide 8 (CYP2C8), transcript variant Hp1-1,
mRNA
gi|13787188|ref|NM_000770.2|[13787188]
NM_030878
Homo sapiens cytochrome P450, family 2, subfamily C, polypeptide 8 (CYP2C8), transcript variant Hp1-2,
mRNA
gi|13787186|ref|NM_030878.1|[13787186]
NM_023025
Rattus norvegicus cytochrome P450, family 2, subfamily J, polypeptide 4 (Cyp2j4), mRNA
gi|61889087|ref|NM_023025.2|[61889087]
DN992115
TC119679 Human adult whole brain, large insert, pCMV expression library Homo sapiens cDNA clone
TC119679 5′ similar to Homo sapiens cytochrome P450, family 2, subfamily J, polypeptide 2 (CYP2J2),
mRNA sequence
gi|66251946|gb|DN992115.1|[66251946]
Z84061
SSZ84061 Porcine small intestine cDNA library Sus scrofa cDNA clone c13d09 5′
similar to cytochrome P450 monooxygenase CYP2J2, mRNA sequence
gi|1806390|emb|Z84061.1|[1806390]
BC091149
Rattus norvegicus cytochrome P450, family 2, subfamily J, polypeptide 4, mRNA (cDNA clone
MGC: 108684 IMAGE: 7323516), complete cds
gi|60688166|gb|BC091149.1|[60688166]
NW_380169
Bos taurus chromosome Un genomic contig, whole genome shotgun sequence
gi|61630302|ref|NW_380169.1|BtUn_WGA215002_1[61630302]
BC032594
Homo sapiens cytochrome P450, family 2, subfamily J, polypeptide 2, mRNA (cDNA clone MGC: 44831
IMAGE: 5527808), complete cds
gi|21595666|gb|BC032594.1|[21595666]
NT_086582
Homo sapiens chromosome 1 genomic contig, alternate assembly
gi|51460368|ref|NT_086582.1|Hs1_86277[51460368]
NT_032977
Homo sapiens chromosome 1 genomic contig
gi|51458674|ref|NT_032977.7|Hs1_33153[51458674]
CO581852
ILLUMIGEN_MCQ_46633 Katze_MMJJ Macaca mulatta cDNA clone IBIUW: 17960 5′ similar to Bases
384 to 953 highly similar to human CYP2J2 (Hs.152096), mRNA sequence
gi|50413382|gb|CO581852.1|[50413382]
AY410198
Mus musculus CYP2J2 gene, VIRTUAL TRANSCRIPT, partial sequence, genomic survey sequence
gi|39766166|gb|AY410198.1|[39766166]
AY410197
Pan troglodytes CYP2J2 gene, VIRTUAL TRANSCRIPT, partial sequence, genomic survey sequence
gi|39766165|gb|AY410197.1|[39766165]
AY410196
Homo sapiens CYP2J2 gene, VIRTUAL TRANSCRIPT, partial sequence, genomic survey sequence
gi|39766164|gb|AY410196.1|[39766164]
AY426985
Homo sapiens cytochrome P450, family 2, subfamily J, polypeptide 2 (CYP2J2) gene, complete cds
gi|37574503|gb|AY426985.1|[37574503]
AB080265
Homo sapiens CYP2J2 mRNA for cytochrome P450 2J2, complete cds
gi|18874076|dbj|AB080265.1|[18874076]
AF272142
Homo sapiens cytochrome P450 (CYP2J2) gene, complete cds
gi|21262185|gb|AF272142.1|[21262185]
U37143
Homo sapiens cytochrome P450 monooxygenase CYP2J2 mRNA, complete cds
gi|18254512|gb|U37143.2|HSU37143[18254512]
AF039089
Homo sapiens cytochrome P450 (CYP2J2) gene, partial cds
gi|14486567|gb|AF039089.1|AF039089[14486567]
CYP5 Family of Cytochrome P450 Enzymes (sequences from Genbank)
NM_011539
Mus musculus thromboxane A synthase 1, platelet (Tbxas1), mRNA
gi|31981465|ref|NM_011539.2|[31981465]
NM_030984
Homo sapiens thromboxane A synthase 1 (platelet, cytochrome P450, family 5, subfamily A) (TBXAS1),
transcript variant TXS-II, mRNA
gi|13699839|ref|NM_030984.1|[13699839]
NM_001061
Homo sapiens thromboxane A synthase 1 (platelet, cytochrome P450, family 5, subfamily A) (TBXAS1),
transcript variant TXS-I, mRNA
gi|13699838|ref|NM_001061.2|[13699838]
BC041157
Homo sapiens thromboxane A synthase 1 (platelet, cytochrome P450, family 5, subfamily A), transcript
variant TXS-I, mRNA (cDNA clone MGC: 48726 IMAGE: 5755195), complete cds
gi|27371225|gb|BC041157.1|[27371225]
CYP8 Family of Cytochrome P450 Enzymes (sequences from Genbank)
NM_000961
Homo sapiens prostaglandin I2 (prostacyclin) synthase (PTGIS), mRNA
gi|61676177|ref|NM_000961.3|[61676177]
NM_008968
Mus musculus prostaglandin I2 (prostacyclin) synthase (Ptgis), mRNA
gi|31982083|ref|NM_008968.2|[31982083]
D83402
Homo sapiens PTGIS(CYP8) gene for prostacyclin synthase, complete cds
gi|60683846|dbj|D83402.2|[60683846]
BC062151
Mus musculus prostaglandin I2 (prostacyclin) synthase, mRNA (cDNA clone MGC: 70035
IMAGE: 6512164), complete cds
gi|38328177|gb|BC062151.1|[38328177]

It is a preferred embodiment of the invention to use the PUFA substrate in a single enzyme reaction of the invention (a “single enzyme reaction” referring to the conversion of a PUFA to its oxylipin derivative using at least three sequential enzyme additions), where the substrate is provided at a concentration of about 200 μM or less. The inventors have found that above these substrate concentration levels, the yield of oxylipins from the reaction is reduced. Preferably, the substrate is provided at a concentration of between about 10 μM and about 200 μM. More preferably, the substrate is provided at a concentration of between about 50 μM and about 200 μM. More preferably, the substrate is provided at a concentration of between about 100 μM and about 200 μM.

Although the method of the present invention is applicable to the production of any oxylipin from a PUFA, by way of example, the following methods are described for the production of oxylipins from DPAn-6 or from DHA. In one aspect, the invention provides a method to produce 10,17-diHDPAn-6, comprising contacting DPAn-6 with 15-lipoxygenase, wherein the DPAn-6 is contacted with about 8 to 12 sequential additions of 15-lipoxygenase. In another aspect, the invention provides a method to produce 10,17-diHDHA comprising contacting DHA with 15-lipoxygenase, wherein the DHA is contacted with about 8 to 12 sequential additions of 15-lipoxygenase. Preferably, the additions are made about every 25-35 minutes, with every 30 minutes being particularly preferred. In one aspect, the amount of enzyme in each addition is equal. In one aspect, the DPAn-6 or DHA is contacted with about 10 sequential additions of 15-lipoxygenase. In one aspect, the DPAn-6 or DHA is provided at a concentration of about 200 μM or less.

Although examples of the method are provided herein, it is to be understood that these are for purposes of illustration, and that one of skill in the art will be able to substitute various buffers and modify the temperature of the reaction, or concentrations of reagents, in accordance with the aspects of the invention described herein and using the guidance provided herein.

The present invention also encompasses any composition comprising the oxylipins produced by the method of the invention, including a composition comprising an amount of oxylipin that is produced by a single entire run of a method of the invention. The present invention also includes methods of using such oxylipins, for example, for regulating anti-inflammatory, anti-proliferative, neuroprotective and/or vasoregulatory effects in humans and other animals. Such effects are useful for enhancing the general health of an individual, as well as in treating or preventing a variety of diseases and conditions in an individual.

### GENERAL DEFINITIONS

For the purposes of this application, long chain polyunsaturated fatty acids (LCPUFAs) are defined as fatty acids of at least 18 and more carbon chain length, including fatty acids of 20 or more carbon chain length, containing 2 or more double bonds. LCPUFAs of the omega-6 series include, but are not limited to: linoleic acid (LA, 18:2n-6), γ-linolenic acid (GLA; 18:3n-6), di-homo-gammalinoleic acid (C20:3n-6), arachidonic acid (C20:4n-6), docosatetraenoic acid or adrenic acid (C22:4n-6), and docosapentaenoic acid (C22:5n-6). The LCPUFAs of the omega-3 series include: α-linolenic acid (ALA, 18:3n-3), stearidonic acid (STA or SDA; 18:4n-3), eicosatrienoic acid (C20:3n-3), eicosatetraenoic acid (C20:4n-3), eicosapentaenoic acid (C20:5n-3), docosapentaenoic acid (C22:5n-3), and docosahexaenoic acid (C22:6n-3). The LCPUFAs also include fatty acids with greater than 22 carbons and 4 or more double bonds including, but not limited to, C24:6(n-3) and C28:8(n-3).

The terms “polyunsaturated fatty acid” and “PUFA” include not only the free fatty acid form, but other forms as well, such as the triacylglycerol (TAG) form, the phospholipid (PL) form and other esterified forms.

As used herein, the term “lipid” includes phospholipids; free fatty acids; esters of fatty acids; triacylglycerols; diacylglycerides; monoacylglycerides; lysophospholipids; soaps; phosphatides; sterols and sterol esters; carotenoids; xanthophylls (e.g., oxycarotenoids); hydrocarbons; and other lipids known to one of ordinary skill in the art.

For the purposes of this application, “oxylipins” are defined as biologically active, oxygenated derivatives of polyunsaturated fatty acids, formed by oxidative metabolism of polyunsaturated fatty acids. Oxylipins that are formed via the lipoxygenase pathway are called lipoxins. Oxylipins that are formed via the cyclooxygenase pathway are called prostanoids. Oxylipins formed from the 18 carbon fatty acid, stearidonic acid (SDA) are called SDA-derived oxylipins. Oxylipins formed from the 18 carbon fatty acid, γ-linolenic acid (GLA) are called GLA-derived oxylipins. Oxylipins formed from 20 carbon fatty acids (arachidonic acid and eicosapentaenoic acid) are called eicosanoids. Eicosanoids include prostaglandins, leukotrienes and thromboxanes. They are formed either via the lipoxygenase pathway (leukotrienes) or via the cyclooxygenase pathway (prostaglandins, prostacyclin, thromboxanes). Oxylipins formed from 22 carbon fatty acids (docosapentaenoic acid (n-6 or n-3), docosahexaenoic acid and docosatetraenoic acid) are called docosanoids. Specific examples of the GLA-derived and SDA-derived oxylipins are described herein. Specific examples of other oxylipins described above can be found in U.S. Patent Publication No. 2006/0241088 or U.S. Patent Publication No. US-2007/0248586-A1, supra. General reference to an oxylipin described herein is intended to encompass the derivatives and analogs of a specified oxylipin compound.

As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. For example, the reference compound can be a reference docosanoid such as any docosanoid derived from DHA, DPAn-6, DPAn-3 or DTAn-6, and an analog is a substance possessing a chemical structure or chemical properties similar to those of the reference docosanoid.

According to the present invention, reference to an oxylipin “derived from” a specific LCPUFA, such as a “DPAn-6-derived oxylipin” or a “DPAn-6 oxylipin derivative”, or a “DPAn-6 oxylipin analog” by way of example, refers to an oxylipin that has been produced by any method, using the knowledge of the structure of an oxylipin that can be produced using DPAn-6 as a substrate.

The terms “substituted”, “substituted derivative” and “derivative”, when used to describe a compound of the present invention, means that at least one hydrogen bound to the unsubstituted compound is replaced with a different atom or a chemical moiety. Examples of substituents include, but are not limited to, hydroxy, alkyl, halogen, nitro, cyano, heterocycle, aryl, heteroaryl, amino, amide, ester, ether, carboxylic acid, thiol, thioester, thioether, sulfoxide, sulfone, carbamate, peptidyl, PO₃H₂, and mixtures thereof.

Although a derivative has a similar physical structure to the parent compound, the derivative may have different chemical and/or biological properties than the parent compound. Such properties can include, but are not limited to, increased or decreased activity of the parent compound, new activity as compared to the parent compound, enhanced or decreased bioavailability, enhanced or decreased efficacy, enhanced or decreased stability in vitro and/or in vivo, and/or enhanced or decreased absorbtion properties.

In general, the term “biologically active” indicates that a compound has at least one detectable activity that has an effect on the metabolic or other processes of a cell or organism, as measured or observed in vivo (i.e., in a natural physiological environment) or in vitro (i.e., under laboratory conditions).

The oxygenated derivatives (oxylipins) of long chain polyunsaturated fatty acids (LCPUFAs) include mono-, di-, tri-, tetra-, and penta-hydroxy derivatives of the LCPUFAs, and also include the free, esterified, peroxy and epoxy forms of these derivatives. These mono-, di-, tri-, tetra-, and penta-hydroxy derivatives of LCPUFAs are those derivatives that contain 3, 4 or more double bonds, generally at least two of which are conjugated, and one or more non-carboxy, hydroxyl groups. Preferably, these derivatives contain 4-6 double bonds and at least 1-3 non-carboxy, hydroxyl groups, and more preferably, 2 or more non-carboxy, hydroxyl groups.

Oxygenated derivatives of the omega-3 fatty acids EPA and DHA, catalyzed by lipoxygenase or cyclo-oxygenase enzymes, including acetylated forms of cyclooxygenase 2 (COX2), which are capable of down regulating or resolving inflammatory processes, are commonly referred to as “resolvins”, which is a coined term (neologism) that is functional in nature. The “docosatrienes” are a subclass of oxylipins derived from DHA and contain three conjugated double bonds. “Protectin” is another coined functional term for hydroxy derivatives of the omega-3 fatty acid DHA that have a neuroprotective effect.

According to the present invention, the term “docosanoid” specifically refers to any oxygenated derivatives (oxylipins) of any 22-carbon LCPUFA (e.g., DHA, DPAn-6, DPAn-3, or DTAn-6). The structures of such derivatives are described in detail in U.S. Patent Publication No. 2006/0241088, supra. It is noted that while the present inventors recognize that the novel oxylipin derivatives (docosanoids) described in U.S. Patent Publication No. 2006/0241088, supra, that are derived from DPAn-6, DPAn-3 and DTAn-6 might also be considered to be “resolvins” or “protectins” based on similar functional attributes of such oxylipins, for the purposes herein, it is preferred that such oxylipins be generally referenced using the term “docosanoid”, which provides a clear structural definition of such compounds. According to the present invention, the language “docosanoid-like compounds” or “docosanoid analogs” or “docosanoid derivatives” is intended to include analogs of any docosanoids described herein, including any docosanoids that include a C₂₂ fatty acid having at least three olefinic groups (carbon-carbon double bonds). Similar language can also be used to more generally describe analogs and derivatives of any oxylipins as described herein (e.g., oxylipin-like compounds, oxylipin analogs, oxylipin derivatives).

According to the present invention, the term “SDA-derived oxylipin” specifically refers to any oxygenated derivatives (oxylipins) of SDA. The structures of such derivatives are described in detail herein. The term “GLA-derived oxylipin” specifically refers to any oxygenated derivatives (oxylipins) of GLA. The structures of such derivatives are also described in detail herein. The di- and trihydroxy oxylipins from SDA and GLA, and some of the monohydroxy oxylipins from SDA and GLA disclosed herein, have never before been described, to the best of the present inventors' knowledge. As with the docosanoids described above, while the present inventors recognize that the novel oxylipin derivatives of the present invention that are derived from SDA and GLA might also be considered to be “resolvins” or “protectins” based on similar functional attributes of such oxylipins, for the purposes of this invention, it is preferred that the novel oxylipins of the present invention be generally referenced using the term “SDA-derived oxylipin” or “GLA-derived oxylipin”, which provides a clear structural definition of such compounds.

Oxylipins that can be produced using the method of the present invention are described below. This list is exemplary, and the invention is not limited to production of these oxylipins. ARA-derived oxylipins

An oxylipin derived from ARA that is useful in the present invention includes, but is not limited to, 5,15-dihydroxy eicosatetraenoic acid.

### Eicosapentaenoic Acid (EPA)-Derived Oxylipins

Oxylipins derived from EPA that are useful in the present invention include, but are not limited to: 5,15-dihydroxy eicosapentanoic acid (EPA), 8,15-dihydroxy eicosapentanoic acid (EPA), 15-epi-lipoxin A4 (5S,6R,15R-trihydroxy eicosatetraenoic acid) and its intermediate 15R-hydroxy eicosapentaenoic acid (15R-HEPE); Resolvin E1 (5,12,18-trihydroxy EPA) and its intermediates 5,6-epoxy,18R-hydroxy-EPE, and 5S-hydro(peroxy),18R-hydroxy-EPE, and 18R-hydroxy-EPE (18R-HEPE); and Resolvin E2 (5S,18R-dihydroxy-EPE or 5S,18R-diHEPE) and its intermediates.

### Eicosatrienoic Acid (ETrA)-Derived Oxylipins

Oxylipins derived from eicosatrienoic acid that are useful in the invention, include, but are not limited to, 6-hydroxyeicosatrienoic acid; 6,12-dihydroxyeicosanoic acid 11,18-dihydroxy-eicosatrienoic acid and an analog, derivative or salt thereof. Additional eicosanoids derived from eicosatrienoic acid and that may be produced using the method of the present invention include, but are not limited to: 5-hydroxyeicosatrienoic acid; 6-hydroxyeicosatrienoic acid; 8-hydroxyeicosatrienoic acid; 11-hydroxyeicosatrienoic acid; 15-hyrdroxyeicosatrienoic acid; 18-hydroxyeicosatrienoic acid; 6,12-dihydroxyeicosanoic acid 11,18-dihydroxy-eicosatrienoic acid; 8,15-dihydroxyeicosanoic acid; and an analog, derivative or salt thereof.

### Docosahexaenoic Acid (DHA)-derived Oxylipins

Oxylipins derived from DHA that can be produced using the method of the present invention include, but are not limited to: Resolvin D1 (7,8,17R-trihydroxy DHA) and Resolvin D2 (7,16,17R-trihydroxy DHA) along with their S-epimers and their intermediates including: 17S/R-hydroperoxy DHA, and 7S-hydroperoxy,17S/R—OH-DHA, and 7(8)-epoxy-17S/R—OH-DHA; Resolvin D4 (4,5,17R-trihydroxy DHA) and Resolvin D3 (4,11,17R trihydroxy DHA) along with their S-epimers and their intermediates including 17S/R-hydroperoxy DHA, and 4S-hydroperoxy,17S/R—OH DHA and 4(5)-epoxy-17S/R—OH DHA; and Neuroprotectin D1 (10,17S-docosatriene, protectin D1) along with its R epimer and their intermediates including the dihydroxy product 16,17-epoxy-docosatriene (16,17-epoxy-DT) and the hydroperoxy product 17S-hydroperoxy DHA; Resolvin D5 (7S,17S-dihydroxy DHA) and Resolvin D6 and their hydroxyl containing intermediates; and epoxide derivatives 7,8 epoxy DPA, 10,11-expoxy DPA, 13,14-epoxy DPA, and 19,20-epoxy DPA and dihydroxy derivative 13,14-dihydroxy docosapentaenoic acid; other mono-hydroxy DHA derivatives, including the R and S epimers of 7-hydroxy DHA, 10-hydroxy DHA, 11-hydroxy DHA, β-hydroxy DHA, 14-hydroxy DHA, 16-hydroxy DHA and 17-hydroxy DHA; and other dihydroxy DHA derivatives, including the R and S epimers of 10,20-dihydroxy DHA, 7,14-dihydroxy DHA and 8,14-dihydroxy DHA. DHA-derived oxylipins are described in detail in Serhan, Novel Eicosanoid and Docosanoid Mediators Resolvins, Docosatrienes, And Neuroprotectins, Curr. Opin. Clin. Nutr. Metab. Care, 8(2):115-21 (2005), and Serhan et al, Resolvins, docosatrienes, and neuroprotectins, novel omega-3-derived mediators, and their aspirin-triggered endogenous epimers: an overview of their protective roles in catabasis, Prostaglandins Other Lipid Mediat., 73(3-4):155-72 (2004), and Schwab et al, Lipoxins and new lipid mediators in the resolution of inflammation, Curr. Opin. Pharmacol. 6(4):414-20, 2006, which are incorporated herein by reference.

### DPAn-6-Derived Oxylipins

DPAn-6-derived oxylipins (also referred to as oxylipins, or more particularly, docosanoids, from DPAn-6) include but are not limited to, any R- or S-epimer or an R/S or S/R epimer (or other combination thereof) of any monohydroxy, dihydroxy, trihydroxy, or multi-hydroxy derivative of DPAn-6, and can include hydroxy derivatizations at any carbon that forms a carbon-carbon double bond in DPAn-6. Some exemplary, novel DPAn-6 derived oxylipins that can be produced using the method of the invention include, but are not limited to: the R- and S-epimers, R/S or S/R epimers (or other combination thereof) of the monohydroxy products of DPAn-6, including 7-hydroxy DPAn-6,8-hydroxy DPAn-6,10-hydroxy DPAn-6,11-hydroxy DPAn-6,13-hydroxy DPAn-6,14-hydroxy DPAn-6, and 17-hydroxy DPAn-6 (most particularly 17-hydroxy DPAn-6); the R and S epimers of the dihydroxy derivatives of DPAn-6, including 7,17-dihydroxy DPAn-6, 10,17-dihydroxy DPAn-6, 13,17-dihydroxy DPAn-6, 7,14-dihydroxy DPAn-6, 8,14-dihydroxy DPAn-6, 16,17-dihdroxy DPAn-6, and 4,5-dihydroxy DPAn-6 (most particularly 10,17-dihydroxy DPAn-6); and tri-hydroxy derivatives of DPAn-6, including R and S epimers of 7,16,17-trihydroxy DPAn-6 and 4,5,17-trihydroxy DPAn-6.

### DPAn-3-Derived Oxylipins

DPAn-3-derived oxylipins (also referred to as oxylipins, or more particularly, docosanoids, from DPAn-3) include but are not limited to, any R- or S-epimer, or an R/S or S/R epimer (or other combination thereof) of any monohydroxy, dihydroxy, trihydroxy, or multi-hydroxy derivative of DPAn-3, and can include hydroxy derivatizations at any carbon that forms a carbon-carbon double bond in DPAn-3. Some exemplary, novel DPAn-3 derived oxylipins that can be produced using the method of the invention include, but are not limited to: the R- and S-epimers of the monohydroxy products of DPAn-3, including 7-hydroxy DPAn-3,10-hydroxy DPAn-3,11-hydroxy DPAn-3,13-hydroxy DPAn-3,14-hydroxy DPAn-3,16-hydroxy DPAn-3, and 17-hydroxy DPAn-3; the R and S epimers of the dihydroxy derivatives of DPAn-3, including 7,17-dihydroxy DPAn-3, 10,17-dihydroxy DPAn-3, 8,14-dihydroxy DPAn-3, 16,17-dihydroxy DPAn-3, 13,20-dihydroxy DPAn-3, and 10,20-dihydroxy DPAn-3; and tri-hydroxy derivatives of DPAn-3, including R and S epimers of 7,16,17-trihydroxy DPAn-3.

### DTAn-6-Derived Oxylipins

DTAn-6-derived oxylipins (also referred to as oxylipins, or more particularly, docosanoids, from DTAn-6) include but are not limited to, any R- or S-epimer, or an R/S or S/R epimer (or a combination thereof) of any monohydroxy, dihydroxy, trihydroxy, or multi-hydroxy derivative of DTAn-6, and can include hydroxy derivatizations at any carbon that forms a carbon-carbon double bond in DTAn-6. Some exemplary, novel DTAn-6 derived oxylipins that can be produced using the method of the invention include, but are not limited to: the R- and S-epimers of the monohydroxy products of DTAn-6, including 7-hydroxy DTAn-6,10-hydroxy DTAn-6,13-hydroxy DTAn-6, and 17-hydroxy DTAn-6; the R and S epimers of the dihydroxy derivatives of DTAn-6, including 7,17-dihydroxy DTAn-6, 10,17-dihydroxy DTAn-6, and 16,17-dihydroxy DTAn-6; and tri-hydroxy derivatives of DTAn-6, including R and S epimers of 7,16,17-trihydroxy DTAn-6.

DTrAn-3-Derived oxylipins

Docosatrienoic acid-derived oxylipins (also referred to as oxylipins, or more particularly, docosanoids from docosatrienoic acid) include but are not limited to, any R- or S-epimer, or any R/S or S/R epimer (or a combination thereof) of any monohydroxy, dihydroxy, trihydroxy, or multi-hydroxy derivative of docosatrienoic acid, and can include hydroxy derivatizations at any carbon that forms a carbon-carbon double bond in docosatrienoic acid. Some exemplary, novel docosatrienoic acid derived oxylipins that can be produced using the method of the invention include, but are not limited to: the R- and S-epimers of the monohydroxy products of docosatrienoic acid, including β-hydroxy docosatrienoic acid: 17-hydroxy docosatrienoic acid: 20-hydroxy docosatrienoic acid and 13,14-epoxy, 17-hydroxy docosatrienoic acid.

DDAn-6-Derived Oxylipins

Docosadienoic acid-derived oxylipins (also referred to as oxylipins, or more particularly, docosanoids from docosadienoic acid) include but are not limited to, any R- or S-epimer, or an R/S or S/R epimer (or a combination thereof) of any monohydroxy, dihydroxy, trihydroxy, or multi-hydroxy derivative of docosadienoic acid, and can include hydroxy derivatizations at any carbon that forms a carbon-carbon double bond in docosadienoic acid. Some exemplary, novel docosadienoic acid derived oxylipins that can be produced using the method of the invention include, but are not limited to: the R- and S-epimers of the monohydroxy products of docosadienoic acid, including 17-hydroxy docosadienoic acid; 13,14-epoxy, 17-hydroxy docosadienoic acid, 15,16-epoxy, 17-hydroxy docosadienoic acid; and 13,16-dihydroxy docosadienoic acid.

### Additional C22-PUFA-Derived Oxylipins

Other C22-PUFA-derived oxylipins (also referred to as oxylipins, or more particularly, docosanoids, from a C22-PUFA) include but are not limited to, any R- or S-epimer, or an R/S or S/R epimer (or a combination thereof) of any monohydroxy, dihydroxy, trihydroxy, or multi-hydroxy derivative of C22-PUFAs, and can include hydroxy derivatizations at any carbon that forms a carbon-carbon double bond in the C22-PUFAs. Some exemplary, novel docosanoids that can be produced using the method of the invention include, but are not limited to 4,5-epoxy-17-hydroxy DPA, 7,8-epoxy DHA, 10,11-epoxy DHA, 13,14-epoxy DHA, 19,20-epoxy DHA, 13,14-dihydroxy DHA, 16,17-dihydroxy DTAn-6,7,16,17-trihydroxy DTAn-6,4,5,17-trihydroxy DTAn-6,7,16,17-trihydroxy DTAn-3, 16,17-dihydroxy DTAn-3, 16,17-dihydroxy DTRAn-6,7,16,17-trihydroxy DTRAn-6,4,5-dihydroxy DTAn-6, and 10,16,17-trihydroxy DTRAn-6.

### SDA-Derived Oxylipins

SDA-derived oxylipins (also referred to as oxylipins from SDA) include, but are not limited to, any R- or S-epimer of any monohydroxy, dihydroxy, or trihydroxy derivative of SDA, and can include hydroxy derivatizations at any carbon that forms a carbon-carbon double bond in SDA. Some exemplary, novel SDA-derived oxylipins that can be produced using the method of the invention include, but are not limited to: the R- and S-epimers of the monohydroxy products of SDA, including 6-hydroxy SDA, 7-hydroxy SDA, 10-hydroxy SDA, 12-hydroxy SDA, 15-hydroxy SDA and 16-hydroxy SDA; the R and S epimers of dihydroxy derivatives of SDA, including 6,13-dihydroxy SDA and 6,16 dihydroxy SDA, as well as dihydroxy derivatives with hydroxyl groups at any two carbons at the C6, C7, C9, C10, C12, C13, C15 or C16 positions of SDA; and the R and S epimers of trihydroxy derivatives of SDA, including trihydroxy derivatives with hydroxyl groups at any three of the carbons at the C6, C7, C9, C10, C12, C13, C15 or C16 positions of SDA.

### GLA-Derived Oxylipins

GLA-derived oxylipins (also referred to as oxylipins from GLA) include, but are not limited to, any R- or S-epimer of any monohydroxy, dihydroxy or trihydroxy derivative of GLA, and can include hydroxy derivatizations at any carbon that forms a carbon-carbon double bond in GLA. Some exemplary, novel GLA derived oxylipins that can be produced using the method of the invention include, but are not limited to: the R- and S-epimers of the monohydroxy products of GLA, including 7-hydroxy GLA and 12-hydroxy GLA; the R and S epimers of dihydroxy derivatives of GLA, including 6,13-dihydroxy GLA; and the R and S epimers of trihydroxy derivatives of GLA.

The following experimental results are provided for purposes of illustration and are not intended to limit the scope of the invention.

### EXAMPLES

### Example 1

The following example demonstrates the preparation of 10,17 diHDPAn-6 using the method of the present invention.

1 gm of DPAn-6 (Nuchek Prep, U102A-A26M) was dissolved in ethanol to a concentration of 250 mg/ml. The DPAn-6 stock was added to 15 L of 0.05 M sodium borate buffer, pH 9.0 such that the final concentration of the substrate in the reaction mixture was 200 M. The reaction mixture was divided into 5×3 L (in 4 L beakers) to allow for better stirring and to accommodate foaming. ˜300 mg of Soybean 15-lipoxygenase was dissolved in DDI water at a concentration of 6 mg/ml a few seconds before initiating the enzymatic reaction. 10 ml of this enzyme stock was added to each 3 L reaction. Stirring was continued for 30 minutes at 4° C.

A 50 μl aliquot with 2× dilution was used to estimate amount of conversion of DPA n-6 to derivatives containing conjugated dienes/trienes. 10 ml of freshly prepared enzyme stock (same as above) was added every 30 minutes after acquiring UV scan. 10 enzyme additions were done, after which the reaction was allowed to continue for 30 minutes at 4° C.

300 ml of sodium borohydride stock solution (6.5 mg/ml in 1 M NaOH) was then added to each beaker and stirring was continued at 4° C. for 15 minutes. This was followed by gradual addition of 30 ml glacial acetic acid to each reaction mixture. Extensive foaming was observed. Stirring was continued for another 15 minutes. 10 gm×5 of DCS18 powder was suspended in 100 ml methanol and stirred gently for two minutes. The methanol was decanted and water added. Slurry containing 10 gms of DSC18 was added to each beaker and reaction stirred at 4° C. for 10 minutes to allow for adsorption of the products. The entire mixture was filtered through a large Buchner funnel and then washed with 3 liters of water. The DSC-18 powder was allowed to dry under vacuum for 10 minutes. The powder was scraped off and suspended in 300 ml of 200-proof ethanol and mixed well for two minutes. The mixture was then filtered though a 150 nil, 0.2 filtration unit to remove the DSC-18 powder and referred to as BD-1137-104 and further purified using HPLC techniques.

Each reference described or cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed is:

1. A method to produce oxylipin derivatives of polyunsaturated fatty acids (PUFAs), comprising contacting a PUFA substrate with at least three sequential additions of an enzyme that catalyzes the production of the oxylipin derivatives from the substrate.

2. (canceled)

3. The method of claim 1, wherein the substrate is contacted with at least five sequential additions of the enzyme.

4. (canceled)

5. The method of claim 1, wherein the substrate is contacted with at least fifteen sequential additions of the enzyme.

6. (canceled)

7. The method of claim 1, wherein the substrate is contacted with from about 5 to about 12 sequential additions of the enzyme.

8. The method of claim 1, wherein the substrate is contacted with about 10 sequential additions of the enzyme.

9. (canceled)

10. The method of claim 1, wherein the substrate is contacted with a number of sequential additions of equal amounts of the enzyme sufficient to convert at least 90% of the substrate to an oxylipin.

11. The method of claim 1, wherein the amount of enzyme in each sequential addition of the enzyme is the same.

12. The method of claim 1, wherein the sequential additions of the enzyme occur at equal time intervals.

13. The method of claim 1, wherein each sequential addition of the enzyme is contacted with the substrate about 20 minutes to about 45 minutes after the prior or first addition of enzyme.

14. The method of claim 1, wherein each sequential addition of the enzyme is contacted with the substrate for a time period sufficient to reduce the detectable enzyme activity to about 5% or less.

15. The method of claim 1, wherein the PUFA is selected from the group consisting of: di-homo-gammalinoleic acid (C20:3n-6), arachidonic acid (C20:4n-6), docosatetraenoic acid or adrenic acid (C22:4n-6), docosapentaenoic acid (C22:5n-6), docosadienoic acid (C22:2n-6), eicosatrienoic acid (C20:3n-3), eicosatetraenoic acid (C20:4n-3), eicosapentaenoic acid (C20:5n-3), docosatrienoic acid (C22:3n-3), docosapentaenoic acid (C22:5n-3), docosahexaenoic acid (C22:6n-3), C24:6(n-3), C28:8(n-3), γ-linolenic acid (18:3n-6) and stearidonic acid (18:4n-3).

16. The method of claim 1, wherein the enzyme is selected from the group consisting of a lipoxygenase, a cyclooxygenase, and a cytochrome P450 enzyme.

17. The method of claim 16, wherein the enzyme is selected from the group consisting of: 12-lipoxygenase, 5-lipoxygenase, 15-lipoxygenase, cyclooxygenase-2, hemoglobin alpha 1, hemoglobin beta, hemoglobin gamma A, CYP4A11, CYP4B1, CYP4F11, CYP4F12, CYP4F2, CYP4F3, CYP4F8, CYP4V2, CYP4×1, CYP41, CYP2J2, CYP2C8, thromboxane A synthase 1, prostaglandin 12 synthase, and prostacyclin synthase.

18. The method of claim 1, wherein the PUFA substrate is provided at a concentration of between about 10 μM and about 200 μM.

19. A method to produce 10,17-diHDPAn-6, comprising contacting a PUFA substrate selected from the group consisting of DHA and DPAn-6 with 15-lipoxygenase, wherein the PUFA substrate is contacted with about 8 to 12 sequential additions of 15-lipoxygenase.

20. (canceled)

21. The method of claim 19 or 20, wherein the additions are made about every 25-35 minutes.

22. (canceled)

23. The method of claim 19, wherein the amount of enzyme in each addition is equal.

24. The method of claim 19, wherein the DPAn-6 or DHA is contacted with about 10 sequential additions of 15-lipoxygenase.

25. The method of claim 19, wherein the DPAn-6 or DHA is provided at a concentration of about 200 μM or less.
